(19) United States
(12) Patent Application Publication
(10) Pub. No.: US 2010/0168385 A1
(43) Pub. Date: Jul. 1, 2010

(54) PROCESS FOR PREPARING ENANTIOMERICALLY ENRICHED AMINO-ALCOHOLS

(76) Inventors: Yuan WEI, North Brunswick, NJ (US); Zhongwei TIAN, Zhejiang (CN); Jingyang ZHU, Monmouth Junction, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/642,929

(22) Filed: Dec. 21, 2009

Related U.S. Application Data
(60) Provisional application No. 61/142,014, filed on Dec. 31, 2008.

(51) Int. Cl. C07C 209/44; C07K 1/00

(52) U.S. Cl. 530/345; 564/468

(57) ABSTRACT

A process for preparing an enantiomerically enriched amino alcohol or derivatives thereof having the structure:

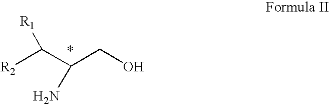

wherein C* is a chiral carbon atom and R₁ and R₂ are independently selected from hydrogen, alkyl, alkoxy, aryl and a peptide chain, wherein said process includes the steps of:
(a) providing an amino acid having the structure of Formula I:

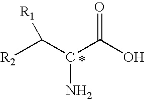

(b) protecting the amino group of the Formula I amino acid with a carbobenzoxy(Cbz) N-protecting group by reacting the amino acid with benzyl chloroformate in the presence of a base;
(c) forming a carboxylic acid alkyl ester of the Cbz N-protected Formula I amino acid by reacting the amino acid with a C₁₋₁₂ alcohol; and
(d) reducing the Cbz N-protected amino acid ester with a borohydride reducing agent in an organic solvent or mixture of organic solvents to form a Cbz N-protected amino-alcohol

This application claims the benefit under 35 U.S.C. 119(e) of U.S. Provisional Application Ser. No. 61/142,014 filed on Dec. 31, 2008.

### BACKGROUND OF THE INVENTION

The present invention relates to a process for preparing high purity optically pure amino-alcohols according to Formula II, and in particular to amino-alcohol having a greater than 99.8% purity.

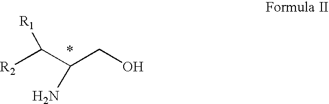

Optically pure amino-alcohols such as L-phenyl-glycinol, phenylalaminol, valinol, methionol, etc., are important intermediates for the pharmaceutical and agrochemical industries. In addition, they provide many therapeutic uses, among which include their ability to inhibit protein synthesis. Chiral amino-alcohols are compounds that possess both amine and alcohol groups. Such structural moieties are believed to provide important biological and pharmacological functions as inhibitors of aspartyl proteases, aldose reductase and b-amyloid peptide formation, and also as dopamine D4 antagonists.

Chiral amino-alcohols have also shown important therapeutic properties as anti-inflammatory, anesthetic, anti-spasmodic, hepatotoxic, anti-diabetic, anti-obesity, and anti-depressant compounds.

The prior art methodologies for preparing amino-alcohols generally employed steps reducing amino acids or esters thereof. In the case of, for example, (S)-valinol, isolated enantiomeric valine or its ester is reacted with a suitable reducing agent such as lithium aluminum hydride in schemes similar to those shown below.

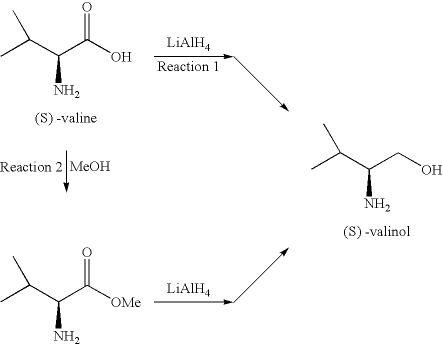

The prior art processes for making enantiomerically enriched valinol suffer from multiple disadvantages, among which one can list the high cost associated with suitable reducing agents, such as lithium aluminum hydride. Lithium aluminum hydride is very expensive, especially in the amounts needed for large scale production. Another disadvantage of prior art methods is the extreme air and moisture sensitive nature of the reactions. Both of the two reactions shown above are also extremely violent, and therefore their use in large scale preparation raises many safety questions.

Attempts have been made to avoid using lithium aluminum hydride as a reducing agent. Accordingly, other prior art methods have used boron hydride reducing agents such as NaBH₄/I, NaBH₄/H₂SO₄, NaBH₄/ZnCl₂, LiBH₄ and BH₃:S(CH₃)₂. For example, in U.S. Pat. No. 3,935,280, amino acids have been reduced to amino-alcohols using boron trifluride and diborane, a borane/ether- or borane/organic sulphides. The relevant amino-alcohol is then obtained in the process after hydrolysis of the reaction mixture.

The shortcoming of such an approach is that valinol easily forms a stable complex with boron. Therefore, after any such complex formation, additional work up must be undertaken to separate valinol from the other components of the complex. Such work up process generally involves heating the product at reflux with strong bases such as KOH for 4-12 h to break down the complex. Since valinol is highly water soluble, isolating it from a water medium creates further complications as it generally requires multiple steps to extract water using suitable organic solvents.

Finally, valinol is relatively unstable towards acid, base and heating, especially heating in an acidic and/or a basic medium, therefore, highly purified and enantiomerically enriched material with >99.8% purity is not achievable by the disclosed prior art methods.

### SUMMARY OF THE INVENTION

The shortcomings of the prior art are now addressed in the present invention. The present invention provides a new process for the preparation of high quality amino-alcohols with greater than 99.8% purity.

The present invention incorporates the discovery that carbobenzoxy N-protected amino acids are not water soluble and do not form stable complexes with boron. This permits the substitution of boron hydride reducing agents for lithium aluminum hydride reducing agents, wherein the resulting carbobenzoxy N-protected amino-alcohols can be easily separated from the boron hydride reducing agents and then subsequently de-protected by palladium catalyzed hydrogenation to yield the high purity amino-alcohol.

The amino-alcohols of the present invention are α-amino-alcohols wherein the amino group is substituted on a carbon atom next to the carbon atom on which the alcohol group is substituted. For purposes of the present invention, unless otherwise specified, “amino-alcohol” means an α-amino-alcohol.

According to one aspect of the present invention a method is provided for preparing an enantiomerically enriched amino-alcohol having the structure:

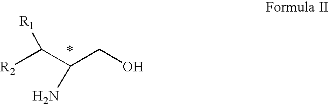

wherein C* is a chiral carbon atom, and R₁ and R₂ are independently selected from hydrogen, alkyl, alkoxy, aryl and a peptide chain, wherein the method includes the steps of;

(a) providing an amino acid having the structure of Formula I:

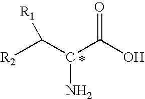

(b) protecting the amino group of the Formula I amino acid with a carbobenzoxy(Cbz) N-protecting group by reacting the amino acid with benzyl chloroformate in the presence of a base;

(c) forming a carboxylic acid alkyl ester of the Cbz N-protected Formula I amino acid by reacting the amino acid with a C₁₋₁₂ alcohol; and

(d) reducing the Cbz N-protected amino acid ester with a borohydride reducing agent to form a Cbz N-protected amino-alcohol

The Cbz N-protected amino-alcohol is not water soluble and unable to form a stable complex with boron. After regular work-up it can be readily extracted from a water medium.

In one embodiment of this aspect of the present invention, the extracted Cbz N-protected amino-alcohol is deprotected by palladium catalyzed hydrogenation in a suitable solvent at a pressure less than 20 bar and a temperature between about −16 and about 100° C. In another embodiment, the amino-alcohol is recovered as a CO₂ complex, from which the amino-alcohol is purified by washing with organic solvent.

In another embodiment, the amino acid starting material may be an (R)-enantiomer, an (S)-enantiomer, a racemic mixture or an enriched racemic mixture. The resulting amino-alcohol with be the corresponding (R)- or (S)-enantiomer, racemate or enriched racemate.

The present invention thus provides an enantiomeric-ally enriched amino-alcohol at purity levels exceeding 99.8%. Advantageously, the process of present invention occurs at a pH wherein the reducing step occurs without employing any acid or bases to compromise the purity of the amino-alcohol.

### DETAILED DESCRIPTION OF THE PREFERRED EMBODIMENTS

As employed above and throughout the disclosure, the following terms, unless otherwise indicated, shall be understood to have the following meanings:

The term “alkyl” refers to the radical of saturated aliphatic groups, including straight-chain C₁₋₁₂ alkyl groups and branched-chain C₁₋₁₂ alkyl groups. The term “alkoxy” as used herein refers to an C₁₋₁₂ alkyl group, as defined above, having an oxygen radical attached thereto. Representative alkoxyl groups include methyloxy, ethoxy, propoxy, butoxy, benzyloxyl, etc.

The term “alcohol” as used herein means an alcohol solvent that comprises a C₁₋₁₂ alkyl moiety substituted at a hydrogen atom with one hydroxyl group. Alcohols include ethanol, n-propanol, i-propanol, n-butanol, i-butanol, s-butanol, t-butanol, n-pentanol, i-pentanol, n-hexanol, cyclohexanol, n-heptanol, n-octanol, n-nonanol, n-decanol, and the like. The carbon atoms in alcohols can be straight, branched or cyclic.

The term “aryl” means an aromatic ring or two or more fused rings that contain one or more aromatic rings where the ring or fused rings may be optionally substituted or substituted with one to two substituents independently selected from amino, hydroxyl, fluoro, C₁₋₄ alkyl, C₁₋₄ alkoxyl, and trifluoromethyl.

The term “protecting group” means a moiety that prevents the atom to which it is linked from participating in unwanted reactions.

The term “peptide chain” refers to two or more amino acids linked through a normal peptide bond, i.e., —CO—NH—, between adjacent amino acid residues. Peptides comprise dipeptides (dimers), tripeptides (trimers), short peptides of 4, 5, 6, 8, 10 or 15 residues, and the like.

Accordingly, the present invention provides a new process for preparing amino-alcohols, including (S)-valinol, which represents an improvement over the prior art. For purposes of illustration, the inventive process will be described for preparing (S)-valinol from (S)-valine. However, one of ordinary skill in the art will understand that essentially any amino acid, and, in particular, any (R)- or (S)-α-amino acid can be substituted for (S)-valine to obtain the corresponding amino-alcohol.

The preparation of (S)-valinol from (S)-valine is elucidated in Scheme 1:

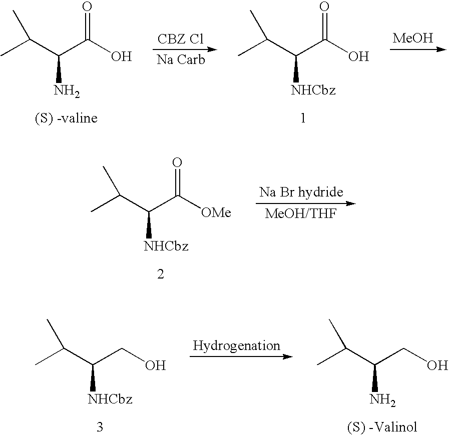

In particular, the present invention includes methods of preparing highly enantiomerically enriched amino-alcohols such as valinol by a process including the steps of:

(1) Reacting a suspension of (S)-valine in an aqueous solution of a base such as Na₂CO₃ with benzyl chloroformate to give Cbz N-protected valine (1).

(2) Forming a Cbz N-protected valine alkyl ester (2) by conducting an esterification reaction in a an alcohol solution. Suitable alcohols include, but are not limited to, methanol, ethanol, propanol, butanol, and the like. The reaction is typically performed in the presence of H₂SO₄.

(3) Reducing the Cbz N-protected valine alkyl ester in an organic solvent or mixed organic solvents with a borohydride reducing agent to provide a Cbz N-protected valinol (3). Sodium, lithium or potassium borohydrides may be used as well combinations of borohydrides with Lewis acids.

Suitable organic solvents include C₁₋₁₂ alcohols, ethers, esters, alkyl benzenes, benzene, and combinations thereof. Because compound (3) is not water soluble and does not form stable complexes with boron, after regular work up it can be readily recovered from aqueous media.

(4) Hydrogenating the Cbz protected valinol in an organic solvent or mixed organic solvents and in the presence of a palladium catalyst at less than 20 bar pressure of hydrogen and a temperature ranging from about −16 to about 100° C. to obtain (S)-valinol (4). Suitable organic solvents include C₁₋₁₂ alcohols, ethers, esters, alkyl benzenes, benzene, and combinations thereof. Suitable palladium catalysts include Pd/C, Pd(OH)₂/C, and the like.

The hydrogenation reaction is preferably conducted in methanol at room temperature and a hydrogen pressure between about one and about five bar. Product (4) is a complex of (S)-valinol with CO₂.

The next step removes the reaction solvent to isolate the crude white solid complex of (S)-valinol with CO₂. The complex is a white solid that can be further purified by treatment of the crude solid with organic solvent or mixed organic solvents, preferably tert-butyl methyl ether (TBME) to remove impurities.

Highly pure free (S)-valinol can be obtained by warming the complex at reduced pressure to remove CO₂, or in organic solvent or mixed organic solvents, preferably methanol, under nitrogen flow. If needed the (S)-valinol can be recrystal-lized from an organic solvent such as hexane to give the material as a needle or distilled under vacuum to obtain a lump solid.

The forgoing examples better elaborate the nature of the invention. All parts and percentages are by weight unless otherwise noted and all temperatures are in degrees Celsius. Solvents were of HPLC grade and used without further purification.

### EXAMPLES

### Example 1

### Formation of (s)—N-cbz-Valine

L-Valine (150 g) was suspended in 1000 mL of water and the mixture was treated with Na₂CO₃ (136 g). CbzCl (240 g) was added and the reaction mixture was stirred overnight. The mix-ture was filtered and extracted with 500 mL AcOEt. The aqueous layer was adjusted to pH 2 and extracted with 2×500 mL AcOEt. The combined organic layers were dried over Na₂SO₄ and concentra-ted under reduced pressure to give 350 g (˜100%) of crude product as a colorless oil.

### Example 2

### Formation of (S)—N-cbz-Valine Ethyl Ester

The (S)—N-CBZ-valine from Example 1 (150 g) was mixed with 1200 mL methanol and H₂SO₄ (15 g) was added. The mixture was stirred overnight and distilled under reduced pressure. The residue was treated with 1000 mL AcOEt and washed with 500 mL water, followed by washing with 400 mL saturated NaHCO₃. The organic layer was dried over Na₂SO₄ and concentrated under reduced pressure to give 151 g of product.

### Example 3

### Formation of(S)—N-CBZ-valinol

The (S)—N-CBZ-valine ethyl ester of Example 2 (150 g) was mixed with 1000 mL THF. NaBH₄ was added. 60 mL Methanol was added slowly over one hour. The mixture was cooled to room temperature and poured into 500 mL water. After adjusting the pH to 2, the mixture was extracted with 1000 mL EtOAc and the organic layer was washed with 500 mL brine and dried over Na₂SO₄. The solvent was distilled under reduced pressure to give the crude product (˜100% yield).

### Example 4

### Formation of (S)-Valinol

The (S)—N-CBZ-valinol of Example 3 (100 g) was mixed with 1000 mL methanol in a pressure vessel. Pd/C (5 g, 5%) was added and the reaction mixture was hydrogenated under 3 bar pressure hydrogen. After the reaction was completed the Pd/C was filtered off and solvent removed under reduced pressure to give (S)-valinol/CO₂ as a white solid, which was purified by treating with TBME and filtered. The purified complex was heated under reduced pressure and distilled under reduced pressure using an oil pump to give product with greater than 99.8% purity.

While the invention has been disclosed in connection with the preferred embodiments and methods of use, it is to be understood that many alternatives, modifications, and variations thereof are possible without departing from the present invention. Thus, the present invention is intended to embrace all such alternatives, modifications, and variations as may be apparent to those skilled in the art and encompassed within the hereinafter appended claims.

What is claimed:

1. A process for preparing an enantiomerically enriched amino alcohol or derivatives thereof having the structure: 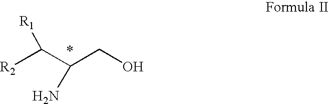
wherein C* is a chiral carbon atom and R₁, and R₂ are independently selected from the group consisting of hydrogen, alkyl, alkoxy, aryl and a peptide chain, wherein said process comprises the steps of:
(a) providing an amino acid having the structure of Formula I: 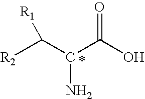
(b) protecting the amino group of said Formula I amino acid with a carbobenzoxy(Cbz) N-protecting group by reacting said amino acid with benzyl chloroformate in the presence of a base;
(c) forming a carboxylic acid alkyl ester of said Cbz N-protected Formula I amino acid by reacting said amino acid with a C₁₋₁₂ alcohol; and
(d) reducing said Cbz N-protected amino acid ester with a borohydride reducing agent in an organic solvent or mixture of organic solvents to form a Cbz N-protected amino-alcohol

2. The method of claim 1, wherein the compound of Formula I is (S)-valine.

3. The method of claim 1, wherein said borohydride reducing agent is sodium borohydride.

4. The method of claim 1, wherein said Cbz N-protected amino alcohol is recovered from said solvent or mixture of solvents essentially free of boron compounds.

5. The method of claim 1, further comprising the step of de-protecting said Cbz N-protected amino-alcohol by palladium catalyzed hydrogenation in organic solvent or a mixture of organic solvents at less than 20 bar pressure hydrogen and a temperature between about −16 and about 100° C.

6. The method of claim 4, wherein said palladium catalyst is Pd/C or Pd(OH)₂/C.

7. The method of claim 4, wherein said temperature is room temperature and said hydrogen pressure is between about one and about five bar.

8. The method of claim 1, wherein the compound of Formula I is an (S)-amino acid.

9. The method of claim 1, wherein the compound of Formula I is an (R)-amino acid.
